(19) United States
(12) Patent Application Publication
(10) Pub. No.: US 2019/0065698 A1
(43) Pub. Date: Feb. 28, 2019

(54) Mechanistic Predictive Model Of Pharmacokinetic Profile Of Oral Compounds In Fasted And Fed State

(71) Applicant: Northeastern University, Boston, MA (US)

(72) Inventors: Rebecca Carrier, Needham, MA (US); Oljora Rezhdo, Cambridge, MA (US); Lauren Speciner, Somerville, MA (US); Selena DiMaio, Boston, MA (US); Fulden Buyukozturk, Boston, MA (US)

(21) Appl. No.: 16/114,725

(22) Filed: Aug. 28, 2018

Related U.S. Application Data
(60) Provisional application No. 62/551,219, filed on Aug. 28, 2017.

(51) Int. Cl. G06F 19/00; G16H 50/50

(52) U.S. Cl. CPC G06F 19/706; G16H 20/10; G16H 50/50; G06F 19/704

(57) ABSTRACT

Models, systems, and methods that accurately predict an impact on drug absorption of foods present in the GI tract are described herein. One example embodiment is a computer-implemented pharmacokinetic model of absorption of an oral compound in a subject. The example computer-implemented model includes memory and a processor in communication with the memory. The memory includes a representation of the subject, a representation of the oral compound, and a representation of a co-administration agent to be consumed by the subject along with the oral compound. The processor is configured to simulate absorption of the oral compound in the subject based on the representation of the subject, representation of the oral compound, and representation of the co-administration agent. The processor is further configured to generate and store in the memory a representation of concentrations over time of the oral compound in blood of the subject. An oral compound and co-administration agent combination can be designed based on a simulation that employs the pharmacokinetic model.

### RELATED APPLICATION

This application claims the benefit of U.S. Provisional Application No. 62/551,219, filed on Aug. 28, 2017. The entire teachings of the above applications are incorporated herein by reference.

### GOVERNMENT SUPPORT

This invention was made with government support under Grant No. RO1GM098117 from the National Institutes of Health. The government has certain rights in the invention.

### BACKGROUND

When a drug (compound) is taken orally, it dissolves in the gastrointestinal (GI) tract. In a fed state, there are many colloidal structures present in the GI tract, such as bile micelles, as well as an aqueous (water) and oil phase into which the drug can partition. This environment is highly dynamic, such that oil and other GI contents are digested, and digestion products can partition into bile micelles, changing their effective volume as well as their chemical nature. Poorly water-soluble compounds have a high affinity for the oil phase, and the center of the bile micelles that have a hydrophobic core, but conventional understanding of drug absorption indicates that only free drug, or dissolved drug in the aqueous phase, is absorbed. In addition, many drug delivery technologies contain compounds or other materials with which a drug interacts, for example by partitioning into that material or complexing with that material (e.g., complexation with cyclodextrins).

### SUMMARY

In prior modeling approaches, compound (e.g., drug) absorption is modeled as driven by the aqueous drug concentration in the intestinal lumen, and these prior approaches do not account for mechanistic impacts of ingested materials, such as, for example, the presence of food in the intestinal lumen and its absorption. Thus, there is a need to accurately predict an impact on drug absorption of ingested materials (e.g., foods, lipids) or other drug delivery materials which a compound interacts. The disclosed embodiments provide such a prediction by modeling mechanistic impacts of consumption of food or drug delivery materials to simulate food (or other ingested material)-drug interactions to account for the impact by ingested materials (e.g., foods, lipids, and complexing agents) on drug absorption more accurately and comprehensively.

One example embodiment is a computer-implemented pharmacokinetic model of absorption of an oral compound in a subject. The example computer-implemented model includes memory and a processor in communication with the memory. The memory includes a representation of the subject, a representation of the oral compound, and a representation of a co-administration agent to be consumed by the subject along with the oral compound. The processor is configured to simulate absorption of the oral compound in the subject based on the representation of the subject, representation of the oral compound, and representation of the co-administration agent. The processor is further configured to generate and store in the memory a representation of concentrations over time of the oral compound in blood of the subject. The processor can also generate and store in the memory a representation of concentrations over time of the oral compound in the intestine of the subject. The representation can also include free oral compound concentrations (solid and in solution) and concentrations of the oral compound associated with other ingested material, such as through a binding or partitioning reaction.

The processor can be configured to determine a bioavailability of the oral compound in the subject based on the representation of concentrations over time of the oral compound in the blood of the subject. The processor can be configured to simulate absorption of the oral compound by simultaneously solving a plurality of mechanism-based differential equations representing gastrointestinal processes of the subject. In such embodiments, the gastrointestinal processes represented by the plurality of mechanism-based differential equations can include oral compound dissolution, oral compound partitioning or binding, oral compound absorption, co-administration agent digestion, co-administration agent dissolution, co-administration agent absorption, co-administration agent partitioning or co-administration agent complexation. The oral compound can be, for example, a drug, nutrient, bacteria, or toxin. The co-administration agent can be, for example, a lipid-based co-administration agent, food, complexing agent, or polymer. The subject can be, for example, a human.

Another example embodiment is a computer-implemented method of evaluating a co-administration agent. The example method includes simulating absorption of an oral compound in a subject based on a pharmacokinetic model accounting for a representation of the subject, a representation of the oral compound, and a representation of the co-administration agent to be consumed by the subject along with the oral compound. The method further includes generating a representation of concentrations over time of the oral compound in blood of the subject to determine how the co-administration agent affects the concentrations of the oral compound in the blood of the subject over time.

The pharmacokinetic model can include a plurality of mechanism-based differential equations representing gastrointestinal processes of the subject. In such embodiments, simulating absorption of an oral compound in the subject based on the pharmacokinetic model can include simultaneously solving the plurality of mechanism-based differential equations representing gastrointestinal processes of the subject. The gastrointestinal processes represented by the plurality of mechanism-based differential equations can include oral compound dissolution, oral compound partitioning or binding, oral compound absorption, co-administration agent digestion, co-administration agent dissolution, co-administration agent absorption, co-administration agent partitioning or co-administration agent complexation. In some embodiments, the method can include determining a bioavailability of the oral compound in the subject based on the representation of concentrations over time of the oral compound in the blood of the subject. As above, the oral compound can be, for example, a drug, nutrient, bacteria, or toxin, the co-administration agent can be, for example, a lipid-based co-administration agent, food, complexing agent, or polymer, and the subject can be, for example, a human.

Another example embodiment is an oral compound (e.g., drug) and co-administration agent combination. The example combination includes an oral compound to be consumed by a subject and a co-administration agent to be consumed by the subject along with the oral compound. The co-administration agent is designed based on a simulation of absorption of the oral compound in the subject based on a pharmacokinetic model accounting for characteristics of the subject, the oral compound, and the co-administration agent. The combination can be a pre-mixed composition, and the co-administration agent can be a lipid-based co-administration agent. The co-administration agent can be designed based on a simultaneous solution of a plurality of mechanism-based differential equations representing gastrointestinal processes of the subject. In such embodiments, the gastrointestinal processes represented by the plurality of mechanism-based differential equations can include oral compound dissolution, oral compound partitioning or binding, oral compound absorption, co-administration agent digestion, co-administration agent dissolution, co-administration agent absorption, co-administration agent partitioning or co-administration agent complexation.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram illustrating a pharmacokinetic model of absorption of an oral compound in a subject, according to an example embodiment.

FIG. 2 is a flow diagram illustrating a computer-implemented method of evaluating a co-administration agent, according to an example embodiment.

FIG. 3 is a schematic diagram illustrating a simulation of drug absorption in a subject that does not account for food (e.g., lipid) absorption.

FIG. 4 is a schematic diagram illustrating a simulation of drug absorption in a subject that accounts for food (e.g., lipid) absorption, according to an example embodiment.

FIG. 5 is a graph illustrating a prediction of a lipid effect on oral pharmacokinetics of a poorly water-soluble drug using different models.

FIG. 6 is a schematic view of a computer network environment in which the example embodiments presented herein may be implemented.

FIG. 7 is a block diagram illustrating an example computer node of the network of FIG. 6.

### DETAILED DESCRIPTION

A description of example embodiments follows.

Disclosed is a computational model (as well as related methods, systems, and combinations) that can predict oral drug pharmacokinetics (PK) of a drug or other orally administered compound (e.g., nutrient, toxin) upon co-administration with a material with which the drug may interact through partitioning or binding, such as food, lipid-based drug delivery systems, or a complexing agent (e.g., cyclodextrin or polymer). The model is a mechanistic model that can predict the effect of food components (e.g., dietary lipids, proteins, carbohydrates, and other food additives) on the absorption of orally dosed compounds in subjects (e.g., humans, as well as other animal models and in vitro systems). The model includes a system of equations that describe dynamic concurrent processes occurring in the gastrointestinal tract during digestion and their direct and indirect effects on oral compound absorption. The model can include processes, such as drug dissolution, food (e.g., lipid) digestion, drug partitioning into/association with different phases in the GI tract (e.g., emulsion droplets, bile micelles), and drug and lipid absorption. These processes can be described by mechanism-based differential equations. The simultaneous solution of these equations can generate an oral compound absorption profile across the intestine in the presence of ingested materials (e.g., foods or lipids). Because absorption is only one of the four components of ADME (Absorption, Distribution, Metabolism, Excretion) that describes drug fate in a body, the model can be paired with compartment-based or physiologic-based pharmacokinetic models to predict oral pharmacokinetics. The model can also serve as a predictive tool in various stages of drug development, from drug discovery to clinical trials, reducing the need for iterative and expensive in vivo testing. The models can also be extended to model the impact of interactions between oral compounds and other material in the intestine that are not ingested (e.g., metabolism by the microbiome, binding to mucins, microbes). Binding to mucins/mucus can drastically change the concentration of free oral compound (which is what drives absorption) in the GI tract.

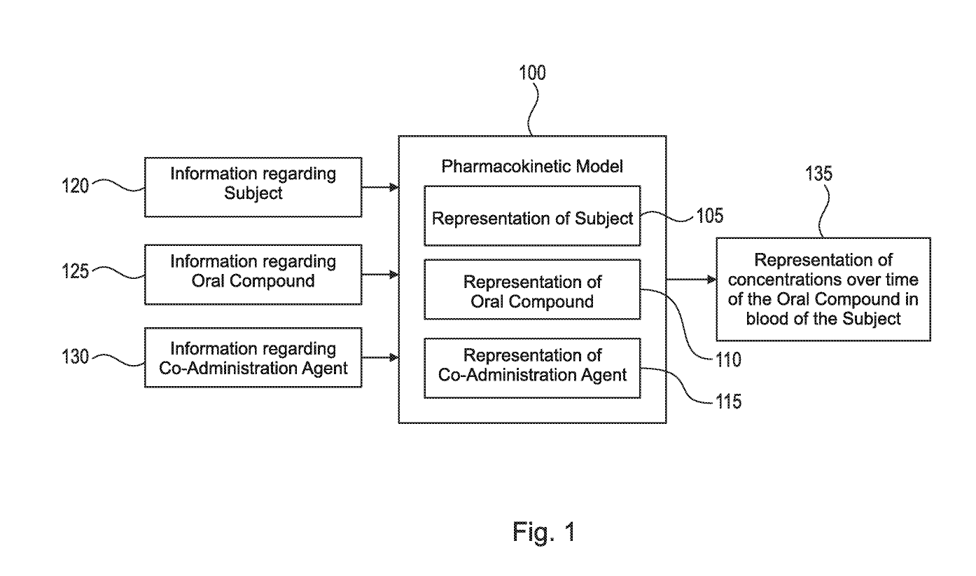

FIG. 1 is a block diagram illustrating a pharmacokinetic model 100 of absorption of an oral compound in a subject, according to an example embodiment. The example model 100 includes a representation of the subject 105, a representation of the oral compound 110, and a representation of a co-administration agent (e.g., food) 115 to be consumed by the subject along with the oral compound. The representation of the subject 105 can be based on information regarding the subject 120, which can be, for example, properties of the intestinal environment of the subject. The representation of the oral compound 110 can be based on information regarding the oral compound 125, which can be, for example, the mass or other properties of the oral compound. The representation of the co-administration agent 115 can be based on information regarding the co-administration agent 130, which can be, for example, the mass or other properties of the co-administration agent. The representations 105, 110, and 115 can be stored in computer memory. A computer processor can be used to simulate absorption of the oral compound in the subject based on the representation of the subject 105, representation of the oral compound 110, and representation of the co-administration agent 115. The processor can generate and store in memory a representation of concentrations over time of the oral compound in blood of the subject 135. In addition, the model can be used to predict concentrations of oral compound in the intestine, and in different forms (e.g., how much solid drug remains, how much drug is in solution, how much drug is partitioned into bile micelles, how much drug is partitioned into lipid droplets).

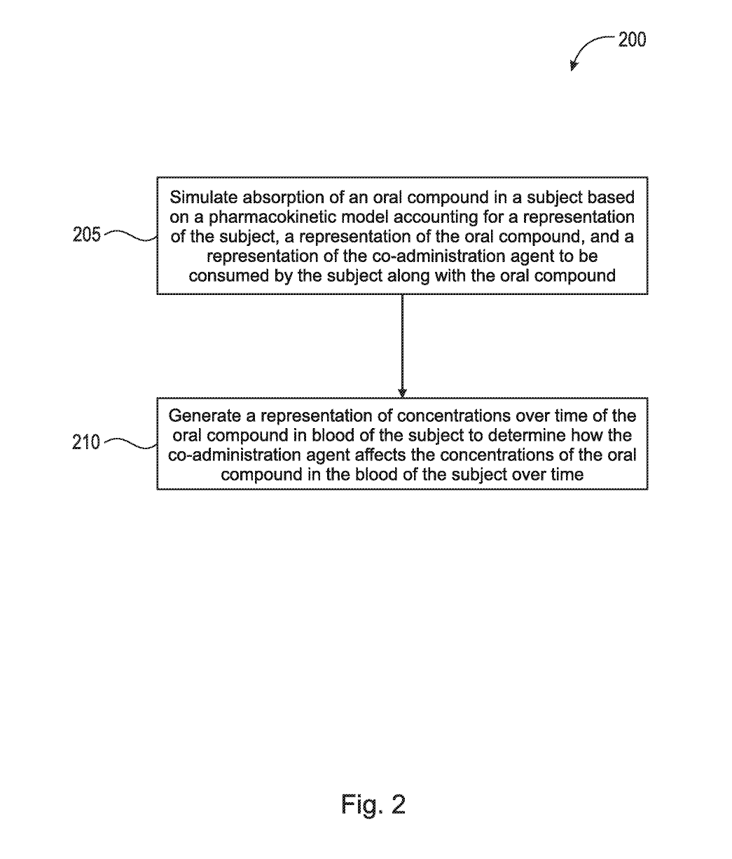

FIG. 2 is a flow diagram illustrating a computer-implemented method 200 of evaluating a co-administration agent, according to an example embodiment. The example method 200 includes simulating 205 absorption of an oral compound in a subject based on a pharmacokinetic model accounting for a representation of the subject, a representation of the oral compound, and a representation of the co-administration agent to be consumed by the subject along with the oral compound. The method further includes generating 210 a representation of concentrations over time of the oral compound in blood of the subject to determine how the co-administration agent affects the concentrations of the oral compound in the blood of the subject over time. The method 200 can simulate absorption of an oral compound by simultaneously solving a plurality of mechanism-based differential equations representing gastrointestinal processes of the subject, such as oral compound dissolution, oral compound partitioning, oral compound absorption, co-administration agent digestion, and co-administration agent absorption.

### Models Not Accounting for Food Presence in the Intestinal Lumen

When a drug is taken orally, it dissolves in the GI lumen. In a fed state, there are many colloidal structures present in the GI tract (such as bile micelles) as well as an aqueous (water) and oil phase into which the drug can partition. This environment is highly dynamic, such that oil and other GI contents are digested, and digestion products can partition into bile micelles, changing their effective volume as well as their chemical nature. Poorly water-soluble compounds have a high affinity for the oil phase, or the center of the bile micelles that have a hydrophobic core. Conventional general understanding of absorption indicates that only free drug, or dissolved drug in the aqueous phase (as opposed to drug within micelles), is absorbed. Therefore, it is useful to understand how the drug interacts with colloidal structures in the GI tract over time to best determine drug absorption, which, in turn, affects the overall PK profile of the drug. Explicitly taking into account these interactions is a focus of the models, methods, systems, and combinations disclosed herein (such as, for example, the model illustrated in FIG. 1).

In prior modeling approaches, which are commonly employed in the pharmaceutical literature and also form the basis for commercially available software programs widely used in the pharmaceutical industry, dissolution is mathematically characterized as being governed by passive Fickian diffusion away from a dissolving particle surface, such that the driving force for diffusion is the concentration gradient between the surface (commonly assumed to be the drug solubility) and the bulk intestinal lumen. The impact of food (e.g., lipids) in these models might be taken into account by altering the solubility in the dissolution equation (shown below) to reflect the solubility in fed state intestinal contents, often approximated by experimentally measuring solubility in simulated intestinal contents with levels of model bile micelle-forming components similar to those expected in the fed state intestine (which are higher than those in a fasted state intestine), and sometimes also containing lipid mixtures simulating a partially digested lipid substance. An empirical correlation, the Glomme correlation, has been used to relate the amount of bile in the intestinal lumen to the aqueous drug solubility (c_{ag}^{eq}). In addition, the diffusion coefficient is commonly modified to reflect the fact that some drug is in micelles in the intestinal lumen, so a weighted diffusion coefficient between what would be anticipated for free drug and for drug within a micelle is used. Thus, this approach implicitly takes into account drug partitioning into micelles in the intestine, a process that is generally considered to be fast (instantaneous) relative to other processes of interest occurring in the intestine (e.g., dissolution and absorption) and characterized by a micelle-water partition coefficient as shown below. Finally, a larger luminal volume (V) is available in the fed state due to the enhanced biliary secretions and the presence of food, and is commonly used in current predictions of the impact of food on oral drug absorption.

In prior modeling approaches, drug absorption is modeled as driven by the aqueous drug concentration in the intestinal lumen. Drug dissolution, drug partitioning, and drug absorption are termed processes as used throughout this description. In current commercially available computational predictive models, equations describing these processes are incorporated into a system of differential equations, the simultaneous solution of which attempts to generate the profile of a drug absorbed over time.

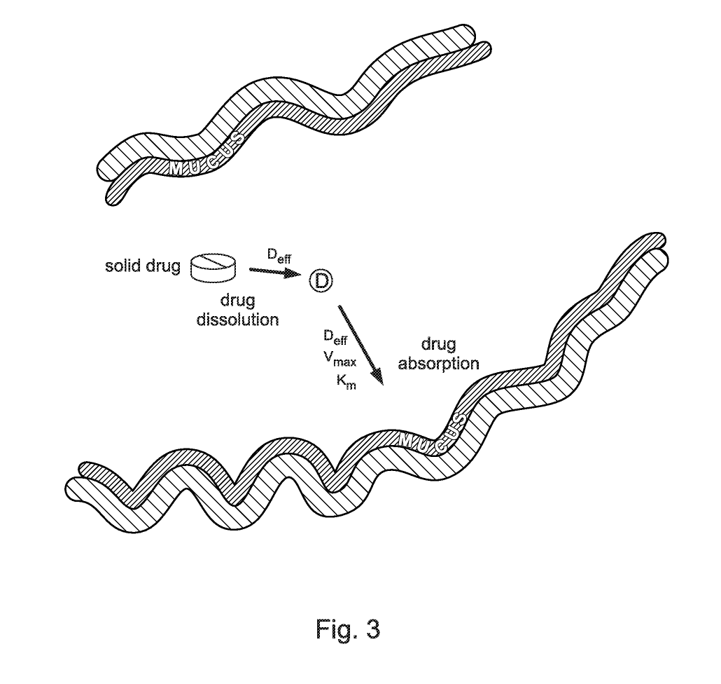

FIG. 3 is a schematic diagram illustrating a simulation of drug absorption in a subject that does not account for food (e.g., lipid) absorption. Input parameters (either known or determined experimentally) include:

Drug amount

Solid drug particle radius

Aqueous drug solubility (c_{aq}^{eq})

Micelle-water partition coefficient (K_{m/b})

Effective intestinal drug permeability (P_{eff})

Intestinal surface area (A)

Effective thickness of the boundary layer (h_{eff})

Effective diffusivity of the dissolved drug (D_{eff})

The drug dissolution equation can be expressed as:

$$\frac{{dC}_{aq}}{dt} = \frac{{D}_{eff} S}{{Vh}_{eff}} ( {C}_{aq}^{eq} - {C}_{aq} )$$

where S is the surface area of the dissolving solid drug particle, D_{eff} is the effective diffusivity of the dissolved drug into the aqueous environment, h_{eff} is the effective thickness of the boundary layer surrounding the solid particle, V is the volume of the dissolution compartment, and C_{aq}^{eq} and C_{aq} are the drug solubility and drug concentration in the aqueous bulk lumen, respectively.

The drug partitioning equation can be expressed as:

$${K}_{m / b} = \frac{{C}_{micelles}}{{C}_{buffer}}$$

$$and$$

$${C}_{aq} = {C}_{micelles} + {C}_{buffer}$$

where C_{micelles} is the drug concentration in micelles defined as the amount of drug in micelles in a unit volume, and C_{buffer} is the drug concentration in the buffer phase defined as the amount of drug in buffer per unit buffer volume.

The drug absorption equation can be expressed as:

$$\frac{{dm}_{abs}}{dt} = {AP}_{eff} {C}_{aq} + \frac{{V}_{max} {C}_{aq}}{{K}_{m} + {C}_{aq}} V$$

where m_{abs} is the mass of absorbed drug, P_{eff} is the effective intestinal permeability due to passive diffusion, A is the intestinal surface area available for absorption, V is the luminal volume, Vₘₐₓ and Kₘ Michaelis-Menten coefficients that can be used to describe active processes such as active transport, efflux, or metabolism, and C_{aq} is the aqueous drug concentration in the lumen.

The simultaneous solution of the equations describing the processes described above predicts the absorption profile of a solid drug in the intestine. When complemented with drug-specific pharmacokinetic parameters (e.g., distribution, clearance) and a compartmental framework or other appropriate pharmacokinetic model (e.g., physiologically-based pharmacokinetic model considering drug concentrations in different organs or other body compartments), one can predict the pharmacokinetic profile of the orally delivered drug in fasted and fed states.

### Models Accounting for Presence of Food or Other Materials that Interact with Drug in the Intestine

Prediction of the absorption profile and ultimately PK profile in the fed state, using prior models is often incorrect, primarily because the mechanisms of food-drug interactions and the dynamic nature of the GI tract during food/lipid digestion are not captured in conventional equations and models. Similarly, prior models are not able to accurately predict the impact of lipid-based drug delivery systems or other delivery systems that interact with drug (e.g., complexation agents such as cyclodextrins) because they do not explicitly consider interactions with these agents. In addition, depending on the drug, the amount and type of food/delivery agent consumed (e.g., high fat vs. low fat) can affect the PK profile. These effects are not captured in commercially available software or published models. Provided herein is a mechanistic and predictive model that describes food (e.g., lipid)-drug interactions, and respective example equations to describe the processes in the model, which, when solved simultaneously, can more accurately and comprehensively predict the impact of food and lipids on oral compound absorption.

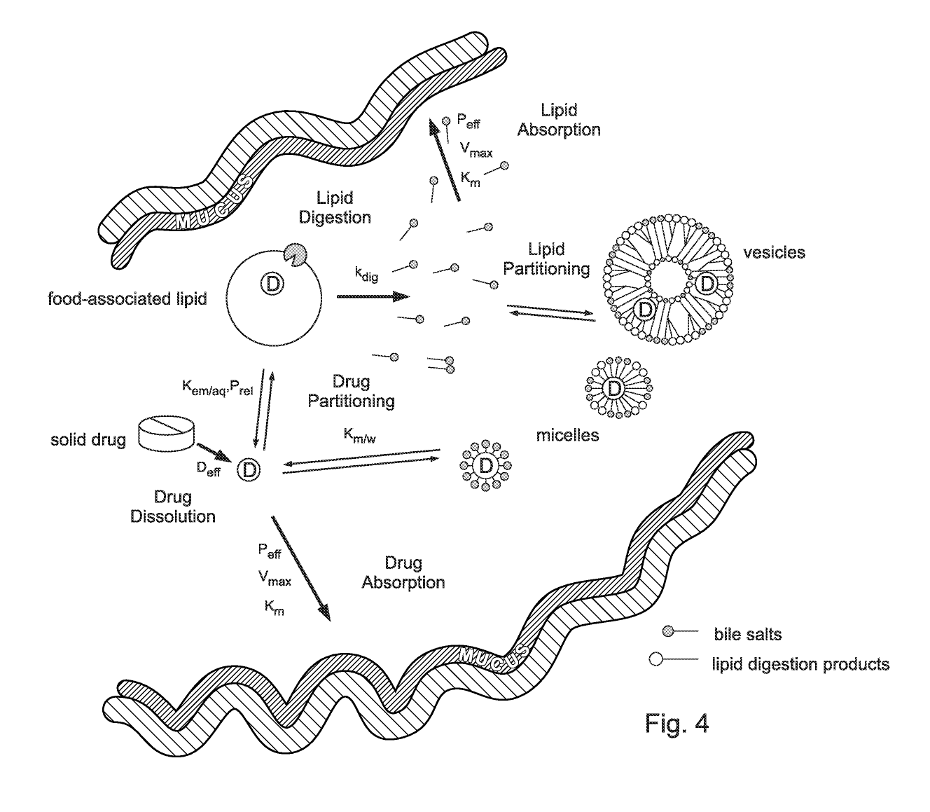

As lipids are a major food component and can also be used to design lipid-based delivery systems, and poorly water-soluble drugs (constituting the majority of marketed oral drugs) have an affinity for hydrophobic/lipophilic environments, the disclosed mechanism and analysis describe lipid-drug interactions and their impact on oral compound absorption. After the ingestion of food, several organs secrete digestion enhancing agents. The pancreas releases lipase, an enzyme used to break down ingested lipids. For example, an ingested triglyceride (the most common type of lipid found in food) is digested into diglycerides (DG), monoglycerides (MG), and fatty acids (FA). A solid drug that has been taken orally dissolves in the aqueous fluids of the intestine and can partition into hydrophobic microenvironments within micelles and oil droplets. Thus, the concentrations of drug in the free aqueous phase as well as in micelles in the intestinal lumen are accounted for in the disclosed model. In addition, an equation to account for the diffusion of drug from the aqueous environment into the oil phase is included. Moreover, digestion of lipids tends to deplete the oil phase but enhance the micellar phase, as the products of lipid digestion are bile-like in nature. These digestion products (MG and FA) partition into bile micelles to form “mixed bile micelles” of enhanced volume. Hence, lipid digestion can be mathematically modeled to account for the change in size (and volume) of the two hydrophobic phases (oil droplets and micelles) during digestion. Specifically, the size of the oil phase is dependent on the amount of triglycerides present in solution over time, whereas the size of the micellar phase is dependent on the amount of fatty acids and monoglyceride present in solution over time. Diglycerides are intermediate compounds and do not largely contribute to the micellar phase. FIG. 4 illustrates schematically these interactions.

FIG. 4 is a schematic diagram illustrating a simulation of drug absorption in a subject that accounts for a co-administration agent (e.g., food, such as a lipid) absorption, according to an example embodiment. Additional input parameters that can be used to account for a co-administration agent (e.g., lipid) with respect to the model of FIG. 3 can include:

Oil amount

Oil droplet radius

Oil permeability (Pᵣₑₗ)

Lipid digestion rate constants (K, K′, K_{I}, K_{E})

An oil-water partitioning equation can be expressed as:

$$\frac{{dC}_{oil}}{dt} = - \frac{{A}_{oil} {P}_{rel}}{{V}_{em}} ( {K}_{oil / aq} {C}_{aq} - {C}_{oil} )$$

where Aₒᵢₗ is the total surface area of the oil droplets, Pᵣₑₗ is the relative drug permeability at the oil-water interface, C_{aq} and Cₒᵢₗ is the drug concentration in the aqueous medium and in the oil phase, respectively, and K_{oil/aq} is the drug partition coefficient between the oil droplets and the micelle-rich aqueous environment.

Lipid digestion equations (as an example; different lipid digestion models, as well as digestion models for other food components, can be used) can be expressed as:

$$\frac{{dC}_{TG}}{dt} = \frac{- {KC}_{TG} {q}_{E , tot}}{2 σ}$$

$$\frac{{dC}_{DG}}{dt} = \frac{K ( {C}_{TG} - {C}_{DG} ) {q}_{E , tot}}{2 σ}$$

$$\frac{{dC}_{MG}}{dt} = \frac{K ( {C}_{DG} - 0.94 {C}_{MG} ) {q}_{E , tot}}{1.94 σ}$$

$$\frac{{dC}_{FA}}{dt} = \frac{K ( {C}_{TG} + {C}_{DG} + 0.94 {C}_{MG} ) {q}_{E , tot}}{1.85 σ}$$

$$where$$

$$σ = 1 + \frac{{A}_{oil}}{{V}_{bulk}} {KK}^{′} ( {C}_{TG} + {C}_{DG} + 0.94 {C}_{MG} ) + \frac{\frac{0.85 {V}_{bulk}}{{A}_{oil} {C}_{FA}}}{{K}_{I}}$$

$$and$$

$${q}_{E , tot} = \frac{{V}_{bulk} {C}_{E , 0}}{{A}_{oil} + \frac{{V}_{bulk}}{{K}_{E}}}$$

C_{TG}, C_{DG}, C_{MG}, C_{FA}, and C_{E,O} are the concentrations of the TG, DG, MG, FA, and enzyme, respectively. K, K′, K_{I}, and K_{E} are digestion rate constants.

Digestion-dependent volume equations can be expressed as:

Volume of oil droplets:

$$\frac{{V}_{oil}}{{V}_{bulk}} = \frac{{MW}_{TG} {C}_{TG}}{{ρ}_{TG}}$$

where Vₒᵢₗ is the total volume of the oil phase, V_{bulk} is the solution volume, MW_{TG} is the molecular weight of the triglyceride, C_{TG} is the molar concentration of the triglyceride, and ρ_{TG} is the density of the triglyceride.

Volume of micelles:

V_{micelles}=N_{micelles}(V_{0,micelle}+0.15C_{FA}N_{A}\tilde{V}_{FA})

V_{micelles} is the total micellar volume per unit solution volume, N_{micelles} is the total number of micelles per unit solution volume, V_{0,micelle} is the volume of one micelle in the absence of FA, C_{FA,micelle} is the concentration of F_{A} molecules present in the micelles as a function of aqueous bulk volume, N_{A} is the Avogadro number, and \tilde{V}_{FA} is the volume of one oleic acid molecule.

The simultaneous solution of the equations describing the processes described above can be used to predict drug absorption and PK profiles in the presence of lipids.

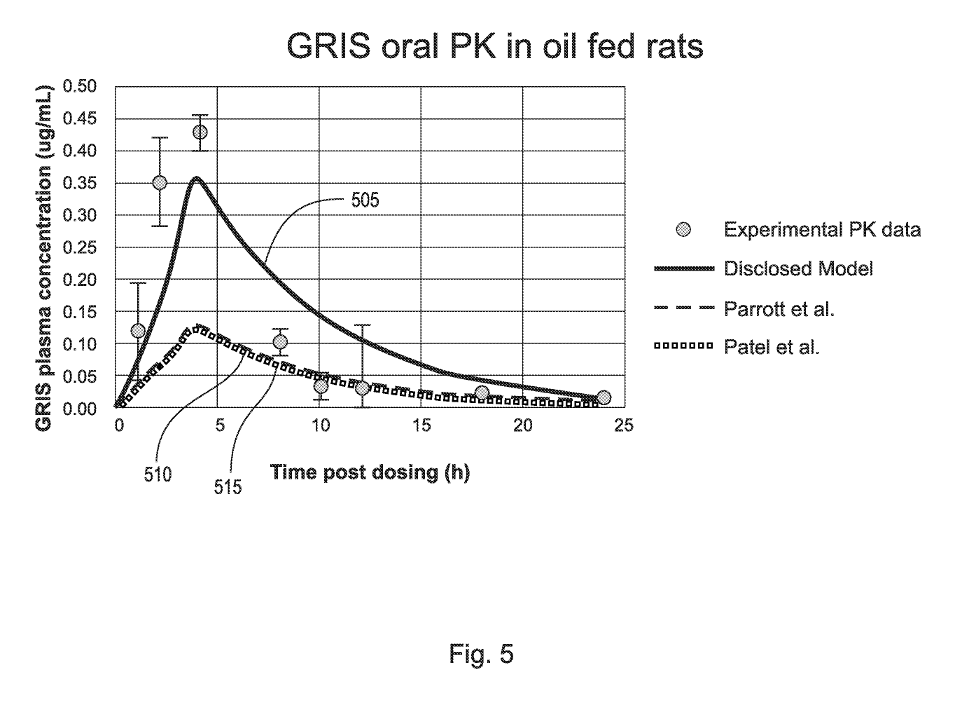

FIG. 5 is a graph illustrating a prediction of a lipid effect on oral pharmacokinetics of a poorly water-soluble drug using different models. In particular, the graph shows prediction of the lipid effect on oral PK of griseofulvin, a poorly water-soluble drug, using equations from the disclosed model 505 versus two commercially available models (“Parrot” 510 and “Patel” 515). The curves for Parrot 510 and Patel 515 are nearly identical. The model disclosed herein more accurately predicts the PK profile because it accounts for the lipid-drug interactions and the changing intestinal environment (e.g., concentration of digestion products and volume of mixed bile micelles) during digestion.

Example features of the models, systems, methods, combinations disclosed herein include the ability to predict drug absorption when co-administered with different formulations and food systems, including different compositions, different amounts, different preparation methods, etc. The disclosed embodiments can take into account mechanistic interactions between ingested/formulation lipids, and other formulation components, and drugs by determining and tracking how the drug partitions through different colloidal species in the gastrointestinal environment and their changes with the digestion process. They can also explicitly account for digestion and absorption of ingested material as well as the impact of lipid absorption on overall system function. They can be used to rationally design lipid-based drug delivery systems, streamlining resources for the drug development process. Alternatively, they can be used to leverage food consumption to enable oral delivery of compounds that may otherwise require injection or to enable lower oral doses of compounds and, thus, minimize costs and potential side effects.

Example advantages of the models, systems, methods, and combinations disclosed herein include the ability to predict food effects more rigorously by using a fully mechanistic model, which eliminates empirical assumptions and, thus, enables broad applicability to different food systems, lipid-based systems, and other drug delivery systems (e.g., complexation agents or polymers with which a drug undergoes molecular interactions) rather than specific applicability to a system being studied. This is in contrast to prior models that often over-predict or under-predict the food effect due to being only semi-mechanistic, i.e., they combine principles of mass transfer with empirical correlations to account for drug dissolution and absorption enhancements due to food. Further, prior models only allow for a binary (fed or fasted) PK prediction. The disclosed embodiments offer the ability to take into account the type and quantity of food/formulation components (such as low fat or high fat diet). While prior models may allow for adjustment of GI input parameters for different animal models or patient populations, the semi-empirical nature makes these prior models prone to additional errors when accounting for effects of these changes on PK predictions. Due to the mechanistic nature of the disclosed embodiments, they can better predict differences in oral PK between different animal models and patient populations. For example, continuous bile secretions in rats versus bolus secretions in humans would largely impact drug-lipid interactions in the rat versus human intestine. The disclosed embodiments can account for such interactions and provide better correlations into predictions of human PK. Due to the mechanistic nature of the disclosed models, systems, and methods, and the fact that interactions between drugs and other entities, including drug delivery systems, can be explicitly accounted for in a model, the model can be extended to other types of food and formulation systems that are not only lipid based.

There are also many example commercial applications of the models, systems, and methods disclosed herein. It has been estimated that more than 40% of new molecular entities discovered have poor water solubility and high lipid solubility. Oral delivery may not be feasible without utilization of a solubilization technology (e.g., amorphous dispersions in polymers, cyclodextrins, lipid-based delivery systems), or exploitation of the impact of food on oral absorption. Lipid-based drug delivery systems have shown great promise for enhancing absorption and bioavailability of hydrophobic compounds. In addition, many hydrophobic compounds show significant “food effects,” or changes in bioavailability when dosed with food, an in particular with high fat content meals. Pharmaceutical companies that discover drugs preferring an oral route of administration need to understand the effect of food on their drug. During clinical trials, the FDA requires testing to determine if there is a food effect on PK. If there is, the drug is generally prescribed to be taken in a fasted state. Hence, the effect of food to enhance oral PK is not being exploited. To date, very few companies use a food effect to their benefit to reduce the dose and unwanted toxicity of their drugs or to change route of administration to take advantage of these benefits.

The disclosed embodiments can provide an in silico environment for a company to test for food effect very early in the discovery process. They can help change route of administration of a new drug to a preferred oral route, or may give guidance on how best to perform clinical trials. In the process, they can reduce the need for extensive animal trials, saving the company time and money. Ultimately, they may be used, if appropriate guidance can be established with the FDA, to replace certain food effect studies and, thus, provide significant savings in resources and time during drug development. The disclosed embodiments can be highly useful to pharmaceutical companies wanting to understand whether compounds they are developing can be delivered orally with acceptable pharmacokinetics if delivered in a lipid-based drug delivery system, with food, or with another agent that interacts with a drug, for example, through a partitioning or binding interaction. Because the disclosed models are intended to predict food effects, they may be able to be used to replace clinical studies used to measure and quantify food effects for orally delivered drugs. The disclosed embodiments can be used by pharmaceutical companies to guide formulation development with lipid-based or food-based formulations. They would be particularly attractive to companies that are currently marketing predictive software widely to the pharmaceutical industry that do not have broad capability for predicting the impact of food or lipids on oral pharmacokinetics. The disclosed embodiments can also be used in the food and nutritive supplement industry, as many supplements on the market are not adequately absorbed on their own. Additionally, with respect to the effect of food on compound absorption, certain compounds may have a negative food effect, which may or may not be desired in certain cases. The disclosed embodiments can be used to predict such effects.

### Example Digital Processing Environment

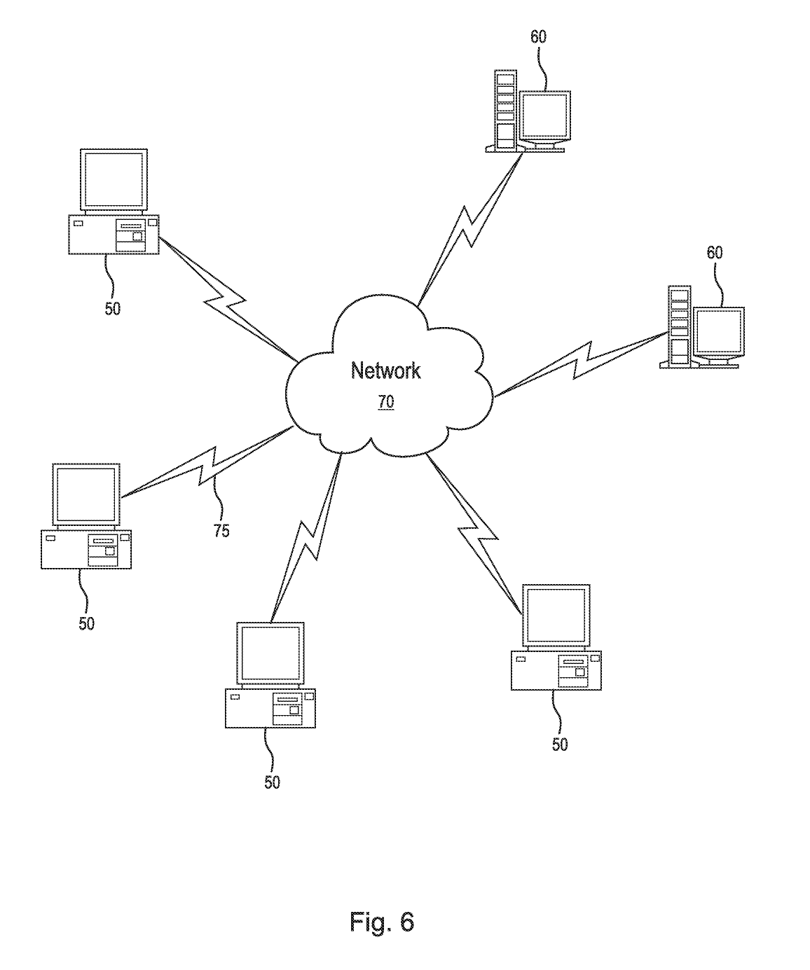

FIG. 6 illustrates a computer network or similar digital processing environment in which the present embodiments may be implemented. Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable. In the context of such a network, a subscription-based service can be implemented on a server, for example, where the connected devices can provide to the service data used by the example systems and methods disclosed herein to predict future performance of products (e.g., book sales).

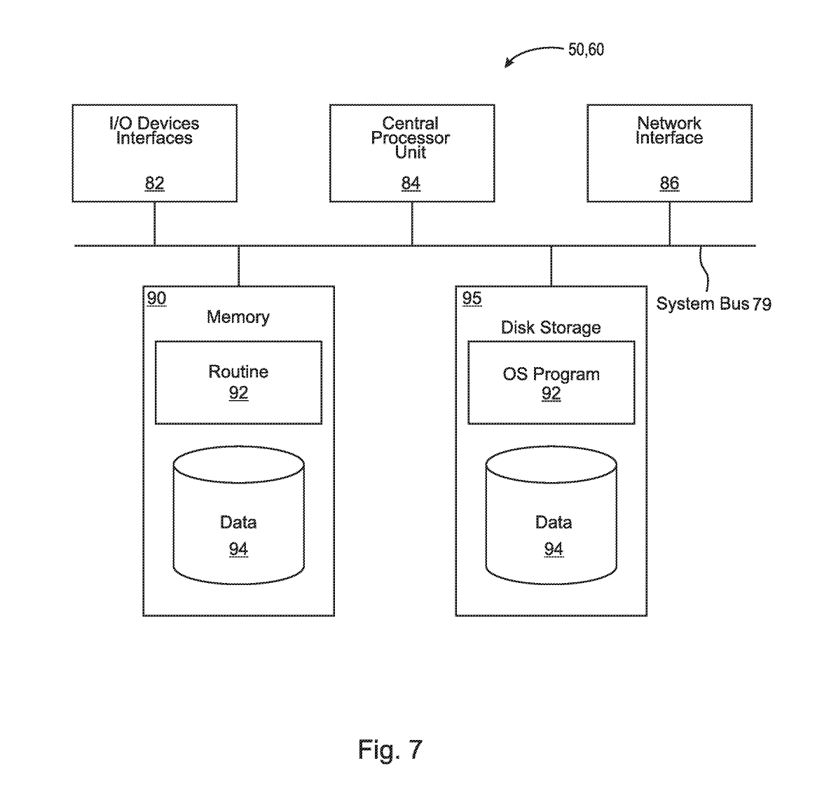

FIG. 7 is a diagram of the internal structure of a computer (e.g., client processor/device 50 or server computers 60) in the computer system of FIG. 6. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, and network ports) that enables the transfer of information between the elements. Attached to system bus 79 is I/O device interface 82 for connecting various input and output devices (e.g., keyboard, mouse, displays, printers, and speakers) to the computer 50, 60. Network interface 86 allows the computer to connect to various other devices attached to a network (e.g., network 70 of FIG. 6). Memory 90 provides volatile storage for computer software instructions 92 and data 94 used to implement many embodiments (e.g., the example model 100 of FIG. 1 and the example method 200 of FIG. 2). Disk storage 95 provides non-volatile storage for computer software instructions 92 and data 94 used to implement many embodiments. Central processor unit 84 is also attached to system bus 79 and provides for the execution of computer instructions.

In the context of FIG. 6, the computer 50, 60 can include a pharmacokinetic model of absorption of an oral compound in a subject. Components of the system include a datastore (e.g., memory) 90, 95 and a processor 84. The datastore 90, 95 can store a representation of the subject, a representation of the oral compound, and a representation of a co-administration agent to be consumed by the subject along with the oral compound. The processor 84 can be configured to simulate absorption of the oral compound in the subject based on the representation of the subject, representation of the oral compound, and representation of the co-administration agent. The processor can be further configured to generate and store in the memory a representation of concentrations over time of the oral compound in blood of the subject.

In one embodiment, the processor routines 92 and data 94 are a computer program product (generally referenced 92), including a computer readable medium (e.g., a removable storage medium such as one or more DVD-ROM's, CD-ROM's, diskettes, and tapes) that provides at least a portion of the software instructions for the system. Computer program product 92 can be installed by any suitable software installation procedure, as is well known in the art. In another embodiment, at least a portion of the software instructions may also be downloaded over a cable, communication and/or wireless connection. In other embodiments, the program product 92 may be implemented as Software as a Service (SaaS), or other installation or communication supporting end-users.

While example embodiments have been particularly shown and described, it will be understood by those skilled in the art that various changes in form and details may be made therein without departing from the scope of the embodiments encompassed by the appended claims.

What is claimed is:

1. A computer-implemented pharmacokinetic model of absorption of an oral compound in a subject, the model comprising:
memory including:
a representation of the subject;
a representation of the oral compound; and
a representation of a co-administration agent to be consumed by the subject along with the oral compound; and
a processor in communication with the memory and configured to simulate absorption of the oral compound in the subject based on the representation of the subject, representation of the oral compound, and representation of the co-administration agent, the processor further configured to generate and store in the memory a representation of concentrations over time of the oral compound in blood of the subject.

2. A model as in claim 1 wherein the processor is configured to simulate absorption of the oral compound by simultaneously solving a plurality of mechanism-based differential equations representing gastrointestinal processes of the subject.

3. A model as in claim 2 wherein the gastrointestinal processes represented by the plurality of mechanism-based differential equations include oral compound dissolution, oral compound partitioning or binding, oral compound absorption, co-administration agent digestion, co-administration agent dissolution, co-administration agent absorption, co-administration agent partitioning or co-administration agent complexation.

4. A model as in claim 1 wherein the processor is configured to determine a bioavailability of the oral compound in the subject based on the representation of concentrations over time of the oral compound in the blood of the subject.

5. A model as in claim 1 wherein the oral compound is a drug, nutrient, bacteria, or toxin.

6. A model as in claim 1 wherein the co-administration agent is a lipid-based co-administration agent, food, complexing agent, or polymer.

7. A model as in claim 1 wherein the subject is a human.

8. A computer-implemented method of evaluating a co-administration agent, the method comprising:
simulating absorption of an oral compound in a subject based on a pharmacokinetic model accounting for a representation of the subject, a representation of the oral compound, and a representation of the co-administration agent to be consumed by the subject along with the oral compound; and
generating a representation of concentrations over time of the oral compound in blood of the subject to determine how the co-administration agent affects the concentrations of the oral compound in the blood of the subject over time.

9. A method as in claim 8 wherein the pharmacokinetic model includes a plurality of mechanism-based differential equations representing gastrointestinal processes of the subject, and wherein simulating absorption of an oral compound in the subject based on the pharmacokinetic model includes simultaneously solving the plurality of mechanism-based differential equations representing gastrointestinal processes of the subject.

10. A method as in claim 9 wherein the gastrointestinal processes represented by the plurality of mechanism-based differential equations include oral compound dissolution, oral compound partitioning or binding, oral compound absorption, co-administration agent digestion, co-administration agent dissolution, co-administration agent absorption, co-administration agent partitioning or co-administration agent complexation.

11. A method as in claim 8 further comprising determining a bioavailability of the oral compound in the subject based on the representation of concentrations over time of the oral compound in the blood of the subject.

12. A method as in claim 8 wherein the oral compound is a drug, nutrient, bacteria, or toxin.

13. A method as in claim 8 wherein the co-administration agent is a lipid-based co-administration agent, food, complexing agent, or polymer.

14. A method as in claim 8 wherein the subject is a human.

15. An oral compound and co-administration agent combination, the combination comprising:
an oral compound to be consumed by a subject; and
a co-administration agent to be consumed by the subject along with the oral compound, the co-administration agent designed based on a simulation of absorption of the oral compound in the subject based on a pharmacokinetic model accounting for characteristics of the subject, the oral compound, and the co-administration agent.

16. A combination as in claim 15 wherein the combination is a pre-mixed composition.

17. A combination as in claim 15 wherein the oral compound is a drug, nutrient, bacteria, or toxin.

18. A combination as in claim 15 wherein the co-administration agent is a lipid-based co-administration agent, food, complexing agent, or polymer.

19. A combination as in claim 15 wherein the co-administration agent is designed based on a simultaneous solution of a plurality of mechanism-based differential equations representing gastrointestinal processes of the subject.

20. A combination as in claim 19 wherein the gastrointestinal processes represented by the plurality of mechanism-based differential equations include oral compound dissolution, oral compound partitioning or binding, oral compound absorption, co-administration agent digestion, co-administration agent dissolution, co-administration agent absorption, co-administration agent partitioning or co-administration agent complexation.
